(19) United States
(12) Patent Application Publication
(10) Pub. No.: US 2003/0032072 A1
(43) Pub. Date: Feb. 13, 2003

(54) Method for the assessment of allergenicity

(75) Inventors: Erwin Ludo Roggen, Lyngby (DK); Steffen Ernst, Kobenhavn (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/254,288

(22) Filed: Sep. 25, 2002

Related U.S. Application Data
(63) Continuation of application No. 09/689,554, filed on Oct. 12, 2000.
(60) Provisional application No. 60/160,543, filed on Oct. 20, 1999.

(30) Foreign Application Priority Data
Oct. 15, 1999 (DK) 1999 01486

(51) Int. Cl. G01N 33/567

(52) U.S. Cl. 435/7.21

(57) ABSTRACT

The invention relates to a method for the assessment of the allergenicity or the toxicity of a test compound and for the screening of at least two compounds simultaneously. The invention also relates to a method of characterizing a cell type for the present invention, as well as an assay kit for the high through-put screening of a compound. By contacting a cell culture of at least one cell type of animal origin with the test compound and measuring cytokine responses, the allergenicity of the test compound can be assessed.

### CROSS-REFERENCE TO RELATED APPLICATIONS

This application is a continuation of application Ser. No. 09/689,554 filed Oct. 12, 2000 and claims, under 35 U.S.C. 119, priority or the benefit of Danish application no. PA 1999 01486 filed Oct. 15, 1999 and U.S. provisional application No. 60/160,543 filed Oct. 20, 1999, the contents of which are fully incorporated herein by reference.

### BACKGROUND OF THE INVENTION

1. Field of the Invention

The invention relates to a method for the assessment of the allergenicity or the toxicity of a test compound and for the screening of at least two compounds simultaneously. The invention also relates is to a method of characterising a cell type for the present invention, as well as an assay kit for the high through-put screening of a compound.

2. Description of Related Art

Many compounds elicit allergic or toxic responses in animals as well as in humans. In keeping with the ever increasing development of new compounds, and the resulting need of testing their potential allergenicity or toxicity, methods for determining allergenicity and toxicity have been developed. Historically, testing of compounds have been performed on animals, and to some extent humans, in lack of available qualified technologies. For obvious reasons there is a public and a professional demand and need to replace in vivo studies with in vitro methods of testing compounds, such as allergenes or toxins.

Prior art attempts to uncover cell types, cellular elements and molecules involved in an allergic response have aided the understanding of the complexity of this reaction chain. One such participating element is cytokines. It is well-known that cytokines play a role as part of the establishment of different immune responses.

Other participants are B and T lymphocytes. B and T cells are the mediators of immunity, however their function is under the control of antigen presenting cells, such as dendritic cells. Everson, M. P. et al. (Journal of Leukocyte Biology, vol. 59, 1996, pp.: 494-498) disclose how different tissues induce the production of different T-cell cytokine profiles. The study suggests that distinct dendritic cell populations are responsible for the induction of T cell proliferation and cytokine production. Studies done by Secrist, H. et al. (J. Exp. Med, vol. 181, 1995, pp.: 1081-1089) describe how the cytokine profile of allergen-specific memory CD4+ T-cells can be modulated by the antigen dose. They found that CD4+ T cells produced high levels of the cytokine interleukin-4 (IL-4) when stimulated with low concentrations of allergen and low levels of IL-4 when stimulated with high concentrations of allergen.

Driscoll, K. E. et al. (Environmental Health Perspectives, vol. 105, 1997, pp.: 1159-1164) describe how inhaled particles, such as noxious particles, elicit inflammation in the lung by effecting the lung epithelium cells. In vitro tests were performed on epithelial cells by stimulating with quartz, causing an increase in levels of the cytokine MIP-2 (macrophage inflammatory protein 2) measured as an increase in mRNA level. The investigators found that the response appeared to be dose related. Another research group have demonstrated in vitro silica-induced mRNA expression of chemokines, such as MIP-2 in alveolar epithelial cells.

Prior art studies have mainly focused on the effect of a known allergen on a particular cell population by observing the mechanistic properties of the cells and the types of cytokines produced.

In patent application WO 99/07880 a method for the identification of human allergens and T-lymphocyte antigens in vitro is disclosed wherein human naive T cells, macrophages/monocytes, immortalized B cells and a test compound are mixed and it is determined whether the test compound induces a response from the T-cells. One of the responses measured is mentioned as being a cytokine response.

T cells recognize a processed antigen through interactions of a T cell receptor, which is a result of clonal recombination, and through a peptide in an MHC molecule, which is individual dependent. This of course represent an uncertainty when assessing whether a test compound is an allergen since the response obtained is correlated to the individual from which the cells originate and not to the test compound per se. Thus, a need for a general test is still present.

### SUMMARY OF THE INVENTION

The present invention relates to a method for the in vitro assessment of the allergenicity of a test compound, comprising the steps of:

(a) obtaining a predetermined and precharacterized cell culture comprising at least one cell type of animal, including human, origin capable of substantially non-specific interaction with the test compound, and of responding with a cytokine expression upon interaction,

(b) contacting the cell culture with the test compound,

(c) defining a specific cytokine profile by determining for at least one predetermined cytokine the cytokine response of the cells exhibiting a substantially non-specific interaction with the test compound, and

(d) correlating the cytokine profile to the allergenicity of the test compound.

By the present invention it is possible to assess the allergenicity of a test compound in vitro by contacting the test compound with a cell capable of eliciting a cytokine response and determine said response, based on the finding that a cytokine response, i.e. the type and level of cytokine secreted by the cell, varies depending on the allergenicity of the test compound, and then correlating the IgE response to allergenicity studies carried out in animals. Allergenicity has its ordinary meaning throughout the present specification, i.e. the ability of evoking an IgE response in animals, including humans.

The present invention further relates to a method wherein the cytokine response is determined in at least two cell cultures, each comprising one cell type, such as a co-culture.

In a further aspect of the invention the method as described above relates to the determination of membrane markers induced by the test compound in cells exhibiting a substantially non-specific interaction with the test compound.

Furthermore the invention describes a combined assay for the determination of the allergenicity and toxicity of a test compound.

Not only will the present invention bring benefit to animals and humans, but it will also allow for the large scale screening of potential allergens and toxins, and thereby minimize operation expenses and time.

Another aspect of the present invention is a method for the simultaneous screening of the allergenicity of at least two test compounds, comprising the steps of:

(a) arranging a specific cell type in at least two separate compartments of a cell culture as defined above,

(b) contacting the separated cell culture compartments individually with a test compound,

(c) defining specific cytokine profiles by determining the cytokine responses of the respective cells exhibiting a substantially non-specific interaction with the test compound,

(d) correlating each cytokine profile to the allergenicity of the test compounds.

In yet another aspect of the present invention a method for determining the toxicity of a test compound is provided, following the steps as described above with respect to allergenicity.

Another aspect of the present invention is a method of characterising a cell type as described above, comprising the steps of:

(a) obtaining a predetermined cell culture comprising one cell type of animal, including human, origin capable of substantially non-specific interaction with the test compound, and of responding with a cytokine expression upon interaction,

(b) identifying the cytokines that can be expressed by the cell culture upon non-specific interaction with the test compound by using a non-specific multivalent inducer,

(c) contacting at least a part of the cell culture with a test compound, determining the level of the identified cytokines obtaining a cytokine profile,

(d) immunizing an animal with the test compound and determining the resulting IgE level of the animal,

(e) correlating the cytokine profile to the IgE level determined, and

(f) repeating step c)-e) until at least a test compound of a high IgE level, a test compound of a low IgE level and a test compound of a medium IgE level have been tested.

A further aspect is an assay kit for high through-put screening of a test compound, comprising,

(a) a cell culture comprising at least one animal, including human, cell type,

(b) a cytokine determinant selected from at least one pair of monoclonal antibodies with specificity for a specific cytokine, at least one cytokine-specific probe for mRNA detection, at least one set of cytokine-specific primers for mRNA or cDNA detection,

(c) an assay device comprising at least two compartments.

Also, an aspect of the invention is the use of the assay for screening of the allergenicity or toxicity of at least two test compounds.

### BRIEF DESCRIPTION OF THE DRAWINGS

FIGS. 1A-D are four tables showing the cytokine responses when epithelial cells are stimulated with lipo-polysaccharide (LPS) for a predetermined incubation time.

FIG. 2 shows the cytokine response for 3 different cytokines as reaction to the protease P modifications, wherein the protease P has been modified with respect to allergenicity.

FIGS. 3A-D are graphs showing the reduction in IgE levels (a), the cytokine response of 3 different cytokines (b-d) in relation to the length of PEG used to modify protease P.

FIGS. 4A-C are graphs showing the correlation between 3 different cytokine levels and the IgE levels when stimulating with protease P.

FIG. 5 is a table showing the IgE levels and the cytokine responses for 3 different cytokines when stimulating with a protease and Lipolase.

### DETAILED DESCRIPTION OF THE INVENTION

The present invention relates to an in vitro test for assessing the allergenicity of a test compound. As described above the allergenicity of a test compound is normally assessed in animal studies whereby the IgE level evoked by the test compound is indicative of the allergenicity of the test compound. However, in practice it is difficult to screen large amounts of test compounds for allergenicity. It is a tedious and expensive task to test compounds through animal studies, and for obvious ethical reasons it is desirable to minimize the use of experimental animals, which in turn is in agreement with the EU directive on animal testing.

The present invention is based on the finding that the cytokine response of a cell capable of substantially non-specific interaction with the test compound and of responding with a cytokine expression upon interaction may be correlated to the IgE response that the same test compound would elicit in animal studies.

The term “non-specific interaction” reflects the fact that some of the cells in the immune defence of the organism interacts non-specifically with foreign substances, non-specifically as opposed to the individual response that other cells, in particular T-cells are eliciting. The cells used according to the invention will elicit the cytokine response either by mere contact with the test compound or by uptake of the compound, such as by pinocytosis of the test compound. Accordingly, by interaction is meant the test compound is at least in contact with the cells and may be taken up by the cells. In the present context the term “non-specific” means that a test compound will not bind to the cell surface and exert its effect through receptors specific for the test compound. This is contrary to T cells which bind the processed antigen, such as an allergen through specific interactions by its T cell receptor. Rather as mentioned earlier the uptake mechanisms by the cells of the invention are not specific for the compounds in question.

The cells used according to the invention, capable of exhibiting a substantially non-specific interaction with the test compound, are preferably epithelial cells irrespective of their location in the organism. In common for epithelial cells are that they are part of the primary defence of an organism against foreign substances.

In one embodiment of the invention the epithelial cells of the invention are being derived from respiratory tract epithelial cells. Epithelial cells of the respiratory tract are directly exposed to airborne components of the exterior environment and a part of the primary defence of the organism against foreign substances inhaled. Epithelial cells are often damaged in individuals suffering from asthma and other allergic airway diseases.

In another embodiment of the invention the epithelial cells are gastro-intestinal tract epithelial cells.

In a further embodiment the at least one cell type is keratinocytes. These cells are the major component of the epidermis, serving the role of protection of the underlying tissue.

Yet another at least one cell type of the invention for cell culturing is dendritic cells. The physiological role of dendritic cells is to capture, process and present antigens, providing lymphocytes with co-stimulatory molecules, and to secrete the proper cytokines to initiate immune responses. It has been found that dendritic cells are capable of eliciting a cytokine response through a non-specific interaction with a test compound.

Also, cells normally considered as part of the more individual and specific part of the immune system, such as macrophages, mast cells, and monocytes may be used according to the present invention, when their ability to elicit a non-specific cytokine response is used.

Macrophages defend the body against invading microorganisms by ingestion via phagocytosis. Macrophages also serve as a scavenger for cleaning up damaged cells and cellular debris.

Yet another group of cells, endothelial cells, may be used according to the invention.

Although the cell type used according to the invention may be from any animal, it is preferred that the at least one cell type is derived from human tissue or a human blood cell.

Furthermore, it is preferred that the cell type used is relevant for the main allergy location of the test compound assessed, i.e. test compound suspected of causing pulmonary allergy are preferably tested in a system using respiratory epithelial cells.

Cytokines are a class of signalling molecules that help to regulate inflammatory processes and play an important role influencing the response to antigens, including allergens. Cytokines contribute to the recruitment of inflammatory an immune cells. They are secreted in small amounts but are extremely potent, they act via receptors and are not produced by unstimulated cells.

The production of cytokines by the cell types according to the invention may be viewed as a primary response against a foreign compound. Upon contact between the cell and the foreign compound the cell may bind the compound by unspecific mechanisms, such as unspecific receptor binding. Following contact, the cells may internalize the foreign compound by pinocytosis or phagocytosis. The further enzymatic break down of the foreign compound intracellularly may trigger the synthesis and secretion of cytokines. In the animal or human body the secreted cytokines may act as messenger molecules effecting various neighbouring cell types including immuno cells, such as T cells and B cells, which in turn trigger a secondary response.

To date a wide variety of cytokines are known, however not all cytokines are produced by all cell types and furthermore, not all cytokines produced by the specific cell type may be secreted as a response to a non-specific interaction with a test compound.

Accordingly, the present invention further relates to a method of characterising the specific cytokines of a predetermined cell type to be determined in relation to assessing the allergenicity of a test compound. The method comprises the steps of

(a) obtaining a predetermined cell culture comprising one cell type of animal, including human, origin capable of substantially non-specific interaction with the test compound, and of responding with a cytokine expression upon interaction,

(b) identifying the cytokines that can be expressed by the cell culture upon non-specific interaction with the test compound by using a non-specific multivalent inducer,

(c) contacting a part of the cell culture with a test compound, determining the level of the identified cytokines obtaining a cytokine profile,

(d) immunizing an animal with the test compound and determining the resulting IgE level of the animal,

(e) correlating the cytokine profile to the IgE level determined, and

(f) repeating step c)-e) until at least a test compound of a high IgE level, a test compound of a low IgE level and a test compound of a medium IgE level have been tested.

Once a cell type has been characterized as described above it is possible to use a cell culture of the cell type in question for assessing the allergenicity of a test compound according to the present invention. Thus, by the term “precharacterized” is meant that the cytokine response from the specific cell type in relation to assessing allergenicity has been determined.

It is preferred that more than one cytokine is being assayed for in the method of assessing allergenicity of a test compound, such as at least two cytokines are being assayed for. An improved prediction is obtained when at least four cytokines are being assayed for in the method according to the invention.

In relation to human lung tissue epithelial cells the cytokines assayed for are preferably Interleukin-6 (IL-6), Interleukin-8 (IL-8), MCP-1 and GM-colony stimulating factor (GM-CSF). For example, according to the invention a high allergenic test compound will elicit a low level of the above mentioned cytokines assayed for, whereas a low allergenic test compound will elicit a high level of IL-8 and MCP-1 and a low level of IL-6. For test compounds in between high and low allergenicity a high level of one or two cytokines is seen, whereas the level of the other cytokines being assayed for is low. Thereby it is possible to quantify the allergenicity of a test compound based on the cytokine profile obtained by the present method.

The number and level of cytokines assessed are factors correlating to the allergenic potency of the test compound.

The cytokine profile may be quantified by a boolean method, i.e. +/−presence above a certain level, or by a direct numerical value of the level.

With respect to human lung tissue epithelial cells the boolean method can be exemplified as in Table 1:

TABLE 1

| Allergenicity/ | | | |
|---|---|---|---|
| Cytokine | IL-8 | IL-6 | MCP-1 |
| High | − | − | − |
| Medium high | + | + | − |
| Medium low | + | + | + |
| Low | + | − | + |

In Table 1 + indicates the presence of the cytokine above a certain level, whereas − indicates the non-presence or presence under the specified level.

It is an object of the present invention to provide for a method wherein a cell culture comprises at least one cell type. However, it may be an advantage to obtain a cytokine response from more than one cell type for each test compound assessed. By using more cell types it is possible to obtain an even more improved method than when using one cell type, due to the various cytokines secreted by the different cell types. A method and an assay using more than one cell type may be arranged in several ways.

Two separate cell cultures comprising different cell types may be contacted with the same test compound and the cytokine profile may be obtained from each cell culture separately. The cytokine profiles may then afterwards be combined and correlated to the allergenicity of the test compound.

It is however preferred when using more than one cell culture, that the cultures are co-cultured. Thereby the natural environment for the cells are mimiced to a greater degree, obtaining results having a higher sensibility.

Accordingly, in an aspect of the invention two cell types are co-cultured. A co-culture according to the invention is a culture wherein the different cell types are sharing the same medium.

In a preferred embodiment of the invention the two cell types of the co-culture are physically separated. The cell types may be separated by a semi-permeable membrane allowing for the passage of small molecules for instance the test compounds, and/or the test substance. The physical set up of the co-culture may be such that one cell type is cultured in one compartment and the other cell type is cultured in a second compartment inserted into the first compartment, i.e. a sub compartment of the first compartment.

The advantage of such a co-culture set-up may be that the second cell type will be effected not only by the test compound but also by the cytokines produced by the cell type of the first cell culture inducing a more natural cytokine response of the second cell type.

In yet another embodiment the test compound may not be able to pass through the membrane. This provides for an embodiment, wherein the cell culture in the second compartment is effected by the cytokines produced in the first cell culture, and not by the actual test compound.

In a further embodiment the test compound is added to a culture of one cell type, upon which the supernatant is transferred to a second culture of a second cell type. In this case the cytokines produced in the first culture along with the test compound will effect the second cell culture.

For all the above culture embodiments the test compound may be added to the medium prior to the medium being added to the cell culture(s).

Preferred combinations of co-cultured cell types are epithelial cell types with non-epithelial cell types, such as a combinations wherein the first cell type is selected from: respiratory epithelial cells, gastrointestinal epithelial cells, keratinocytes, and the second cell type is selected from dendritic cells, macrophages, mast cells, monocytes, and endothelial cells.

Accordingly, in one embodiment of the present invention the first cell type is respiratory epithelial cells and the second cell type is selected from dendritic cells, macrophages, mast cells, monocytes, and endothelial cells.

In a preferred embodiment the first cell type is respiratory epithelial cells and the second cell type is selected from dendritic cells or macrophages.

The cytokine response may be determined by any suitable method, such as by use of a determinant in the form of antibodies towards the cytokines produced or in the form of primers or probes for cDNA or mRNA encoding the cytokines produced.

Accordingly, the extracellular cytokine response may be determined by analysis with enzyme-linked immunosorbent assay (ELISA) using antibodies directed to the cytokines assayed for. Thereby a quantitative determination of the cytokines secreted from the cells as response to the interaction with the test compound is obtained.

In another embodiment the intracellular cytokine response is measured by use of ELISA as described above.

In yet another embodiment the cytokine response is determined by in-situ hybidization technique, preferably using probes directed to mRNA encoding the cytokines to be assayed for.

Also, the cytokine response may be determined by use of polymerase chain reaction (PCR) technique, in particular by using primers for mRNA encoding the cytokines to be assayed for.

The cytokines may be determined by analysis with the commercially available techniques of quantitative PCR technique or in-situ PCR technique.

The essential difference between in-situ PCR and quantitative PCR is the number of samples to be analysed. In order to give quantitative data, in-situ PCR requires a number of identical cell cultures (representing the culture under investigation), each subjected to a different number of PCR cycles, in order to identify the dynamic range of the PCR for that specific cytokine/membrane marker in that specific cell culture.

Quantitative PCR requires one sample per cell culture. In such situation the development of the PCR product is followed over time, thus providing quantitative data (ABI Prism 7700 Sequence Detection System).

In addition to the determination of the cytokine response from the cell type(s) used, it may be advantageous to determine membrane markers induced by the test compound in cells exhibiting a substantially non-specific interaction with the test compound. In this respect it is preferred to determine the membrane markers VCAM-1 or ICAM-1.

In one aspect of the invention the level and number of cytokines assessed are factors correlating to the allergenic potency of the test compound. For the purpose of the present invention the level and number of cytokines assessed are factors correlating to low allergenic test compounds, as well as the level and number of cytokines assessed are factors correlating to high allergenic test compounds.

According to the invention a high allergenic test compound may not elicit any cytokine response, i.e. when compared to the baseline cytokine level. In one embodiment of the present invention a high allergenic test compound may not elicit any cytokine response with respect to the four cytokines tested in cell types described in the invention. In a further embodiment a low allergenic test compound may elicit a cytokine response of at least one cytokine, such as two different cytokines. Yet in another embodiment of the invention a medium high allergen may elicit a response of one cytokine, such as IL8. However, the absolute amount secreted may be far less than the response of a low allergenic molecule as measured in picomole.

The method of assessing the allergenicity of a test compound is particular useful in the screening of various test compounds with respect to their allergenicity.

Accordingly, the present invention relates to a method for the simultaneous screening of the allergenicity of at least two test compounds, comprising the steps of:

(a) arranging a specific cell type in at least two separate compartments of a cell culture as defined above,

(b) contacting the separated cell culture compartments individually with a test compound,

(c) defining specific cytokine profiles by determining the cytokine response of the respective cells exhibiting a substantially non-specific interaction with the test compound,

(d) correlating each cytokine profile to the allergenicity of the test compounds.

The screening method is of course preferably used for more than 2 test compounds simultaneously, such as for about 10 test compounds, or even about 100 test compounds simultaneously. The screening method is especially suitable when testing compounds being modified with respect to their allergenicity.

Such modification of a test compound to affect its allergenicity could be by mutation of a protein allergen in its IgE-specific epitopes. The location of these epitopes can be determined by several techniques such as those disclosed by WO 92/10755 (by U. Løvborg), by Walshet et al, J. Immunol. Methods, vol. 121, 1275-280, (1989), and by Schoofs et al. J. Immunol. vol. 140, 611-616, (1987). A preferred method for identification of epitopes is by screening a random peptide library with antibodies (e.g. IgE antibodies) and the high binding peptide sequences are aligned to identify a consensus sequence. These consensus sequences, in turn are compared with the sequence and 3D structure of a parent protein, which is desired to mutate for reduction of allergenicity, in order to identify the linear and structural epitopes of the parent protein.

When searching for such a protein variant with improved properties, it may be an advantage to establish a library of diversified mutants each having one or more changed amino acids introduced and selecting those variants which show improved properties. This desirable property could be reduced allergenicity expressed by a favorable cytokine response in the methods of this invention.

A diversified library can be established by a range of techniques known to the person skilled in the art (Reetz M T; Jaeger K E, in Biocatalysis—from Discovery to Application edited by Fessner W D, Vol. 200, pp. 31-57 (1999); Stemmer, Nature, vol. 370, p.389-391, (1994); Zhao and Arnold, Proc. Natl. Acad. Sci., USA, vol. 94, pp. 7997-8000, (1997); or Yano et al., Proc. Natl. Acad. Sci., USA, vol. 95, pp 5511-5515, (1998); and Deng S J, et al. Proc. Natl. Acad. Sci., USA, Vol. 92(11), 4992-4996 (1995)).

These include, but are not limited to, ‘spiked mutagenesis’, in which certain positions of the protein sequence are randomized by carring out PCR mutagenesis using one or more oligonucleotide primers which are synthesized using a mixture of nucleotides for certain positions (Lanio T, Jeltsch A, Biotechniques, Vol. 25(6), 958,962,964-965 (1998)). The mixtures of oligonucleotides used within each triplet can be designed such that the corresponding amino acid of the mutated gene product is randomized within some predetermined distribution function. Algorithms exist, which facilitate this design (Jensen L J et al., Nucleic Acids Research, Vol. 26(3), 697-702 (1998)).

Another method of creating a diversified gene library is by using ‘family shuffling’ using a number of different, but homologous, genes as a starting point (Stemmer, Nature, vol. 370, p.389-391, 1994). These genes are fragmented and the fragments used as templates for a PCR reaction, which generates hybrid gene products incoporating sequence elements from several of the parent genes.

As described in the above mentioned references, these approaches may be used in parallel or in series to create directed evolution of a protein backbone to acquire desired properties, such as low allergenicity.

In a preferred embodiment, substitutions are found by a method comprising the following steps: 1) a range of substitutions, additions, and/or deletions are listed encompassing several epitope areas, 2) a library is designed which introduces a randomized subset of these changes in the amino acid sequence into the target gene, e.g. by random mutagenesis, 3) the library is expressed, and preferred variants are selected using the methods of the present invention.

In a more preferred embodiment, the preferred variants are selected in an automated assay system, capable of processing many variants of the diversified library in a high throughput format. In that case, the test compound is typically a protein secreted from cells that can be cultured in a titer-plate format (e.g. bacterial or yeast cells or others). The cell supernatant will, in addition to the test compound, comprise a number of other compounds which may cause a cytokine response different from the baseline. These could be intact cells; cell wall or other organelle fragments from lysed cells; lipopolysaccharides; glycoproteins; small molecules etc. It may be an advantage to prevent these compounds from contacting the cells of the current invention in order to minimize background signaling.

In an even more preferred embodiment, at least two assays are carried out in parallel: one being an assay using the methods of this invention, and the other being a functional assay for the test compound. In the case of a protease test compound, the functional assay may be a protease activity assay. A protease activity assay may be determined using the substrate Suc-Ala-Ala-Pro-Phe-p-nitroaniline. The protease cleaves the bond between the peptide and p-nitroaniline to give a visible yellow colour absorbing at 405 nm. Thus, the substrate and a protease solution are mixed and the absorbance is monitored at 405 nm as a function of time as a measure of the protease activity in the sample. The scope of these embodiments of the invention is by no means limited to protease, which serves only to provide an example.

In a most preferred embodiment, this method is supplemented with additional rounds of screening and/or family shuffling of hits from the first round of screening (J. E. Ness, et al, Nature Biotechnology, vol. 17, pp. 893-896, (1999)) and/or combination with other methods of reducing allergenicity by genetic means (such as that disclosed in Wo 92/10755).

The test compound may be any compound suspected of eliciting an allergenic response in animals, including humans. The allergenic response may be any allergenic response, such as a pulmonary allergenic response caused by inhaled compounds, or a skin allergy caused by skin contact with the allergen, or even gastrointestinal allergy caused by digested allergens.

Accordingly, the test compounds may be any protein, such as a glycoprotein, or a lipoprotein, or a proteolipid, or a phospholipid. The term protein is intended to also include peptides and polypeptides.

In particular the test compound may be an enzyme or an enzyme variant, such as glycosyl hydrolases, carbohydrases, peroxidases, proteases, lipases, phytases, polysaccharide lyases, oxidoreductases, transglutaminases and glycose-isomerases, in particular the following:

Proteases

Proteases (i.e. enzymes classified under the Enzyme Classification number E.C. 3.4 in accordance with the Recommendations (1992) of the International Union of Biochemistry and Molecular Biology (IUBMB)) include proteases within this group.

Examples include proteases selected from those classified under the Enzyme Classification (E.C.) numbers:

3.4.11 (i.e. so-called aminopeptidases), including 3.4.11.5 (Prolyl aminopeptidase), 3.4.11.9 (X-pro aminopeptidase), 3.4.11.10 (Bacterial leucyl aminopeptidase), 3.4.11.12 (Thermophilic aminopeptidase), 3.4.11.15 (Lysyl aminopeptidase), 3.4.11.17 (Tryptophanyl aminopeptidase), 3.4.11.18 (Methionyl aminopeptidase);

3.4.21 (i.e. so-called serine endopeptidases), including 3.4.21.1 (Chymotrypsin), 3.4.21.4 (Trypsin), 3.4.21.25 (Cucumisin), 3.4.21.32 (Brachyurin), 3.4.21.48 (Cerevisin) and 3.4.21.62 (Subtilisin);

3.4.22 (i.e. so-called cysteine endopeptidases), including 3.4.22.2 (Papain), 3.4.22.3 (Ficain), 3.4.22.6 (Chymopapain), 3.4.22.7 (Asclepain), 3.4.22.14 (Actinidain), 3.4.22.30 (Caricain) and 3.4.22.31 (Ananain);

3.4.23 (i.e. so-called aspartic endopeptidases), including 3.4.23.1 (Pepsin A), 3.4.23.18 (Aspergillopepsin I), 3.4.23.20 (Penicillopepsin) and 3.4.23.25 (Saccharopepsin); and

3.4.24 (i.e. so-called metalloendopeptidases), including 3.4.24.28 (Bacillolysin).

Examples of relevant subtilisins comprise subtilisin BPN′, subtilisin amylosacchariticus, subtilisin 168, subtilisin mesentericopeptidase, subtilisin Carlsberg, subtilisin DY, subtilisin 309, subtilisin 147, thermitase, aqualysin, Bacillus PB92 protease, proteinase K, Protease TW7, and Protease TW3, and Bacillus PD498 (WO 93/24623).

Specific examples of such readily available commercial proteases include Esperase®, Alcalase®, Neutrase®, Dyrazym®, Savinase®, Pyrase®, Pancreatic Trypsin NOVO (PTN), Bio-Feed™ Pro, Clear-Lens Pro (all enzymes available from Novo Nordisk A/S).

Examples of other commercial proteases include Maxtase®, Maxacal®, Maxapem® marketed by Gist-Brocades N. V., Opticlean® marketed by Solvay et Cie. and Purafect® marketed by Genencor International.

Examples of protease variants are disclosed in EP 130.756 (Genentech), EP 214.435 (Henkel), WO 87/04461 (Amgen), WO 87/05050 (Genex), EP 251.446 (Genencor), EP 260.105 (Genencor), Thomas et al., (1985), Nature. 318, p. 375-376, Thomas et al., (1987), J. Mol. Biol., 193, pp. 803-813, Russel et al., (1987), Nature, 328, p. 496-500, WO 88/08028 (Genex), WO 88/08033 (Amgen), WO 89/06279 (Novo Nordisk A/S), WO 91/00345 (Novo Nordisk A/S), EP 525 610 (Solvay) and WO 94/02618 (Gist-Brocades N. V.).

The activity of proteases and variants thereof can be determined as described in “Methods of Enzymatic Analysis”, third edition, 1984, Verlag Chemie, Weinheim, vol. 5.

Lipases

Lipases (i.e. enzymes classified under the Enzyme Classification number E.C. 3.1.1 (Carboxylic Ester Hydrolases) in accordance with the Recommendations (1992) of the International Union of Biochemistry and Molecular Biology (IUBMB)) include lipases within this group.

Examples include lipases selected from those classified under the Enzyme Classification (E.C.) numbers:

3.1.1 (i.e. so-called Carboxylic Ester Hydrolases), including (3.1.1.3) Triacylglycerol lipases, (3.1.1.4.) Phosphorlipase A₂.

Examples of lipases include lipases derived from the following microorganisms. The indicated patent publications are incorporated herein by reference:

Humicola, e.g. H. brevispora, H. lanuginosa, H. brevis var. thermoidea and H. insolens (U.S. Pat. No. 4,810,414).

Pseudomonas, e.g. Ps. fragi, Ps. stutzeri, Ps. cepacia and Ps. fluorescens (WO 89/04361), or Ps. plantarii or Ps. gladioli (U.S. Pat. No. 4,950,417 (Solvay enzymes)) or Ps. alcaligenes and Ps. pseudoalcaligenes (EP 218 272) or Ps. mendocina (WO 88/09367; U.S. Pat. No. 5,389,536).

Fusarium, e.g. F. oxysporum (EP 130,064) or F. solani pisi (WO 90/09446).

Mucor (also called Rhizomucor), e.g. M. miehei (EP 238,023).

Chromobacterium (especially C. viscosum).

Aspergillus (especially A. niger).

Candida, e.g. C. cylindracea (also called C. rugosa) or C. antarctica (WO 88/02775) or C. antarctica lipase A or B (WO 94/01541 and WO 89/02916).

Geotricum, e.g. G. candidum (Schimada et al., (1989), J. Biochem., 106, 383-388).

Penicillium, e.g. P. camembertii (Yamaguchi et al., (1991), Gene 103, 61-67).

Rhizopus, e.g. R. delemar (Hass et al., (1991), Gene 109, 107-113) or R. niveus (Kugimiya et al., (1992) Biosci. Biotech. Biochem 56, 716-719) or R. oryzae.

Bacillus, e.g. B. subtilis (Dartois et al., (1993) Biochemica et Biophysica Acta 1131, 253-260) or B. stearothermophilus (JP 64/7744992) or B. pumilus (WO 91/16422).

Specific examples of readily available commercial lipases include Lipolase®, Lipolase™ Ultra, Lipozyme®, Palatase®, Novozym® 435, Lecitase® (all available from Novo Nordisk A/S).

Examples of other lipases are Lumafast™, Ps. mendocian lipase from Genencor Int. Inc.; Lipomax™, Ps. peudoalcaligenes lipase from Gist Brocades/Genencor Int. Inc.; Fusarium solani lipase (cutinase) from Unilever; Bacillus sp. lipase from Solvay enzymes. Other lipases are available from other companies.

Examples of lipase variants are described in e.g. WO 93/01285 and WO 95/22615.

The activity of the lipase can be determined as described in “Methods of Enzymatic Analysis”, Third Edition, 1984, Verlag Chemie, Weinhein, vol. 4, or as described in AF 95/5 GB (available on request from Novo Nordisk A/S).

Oxidoreductases

Oxidoreductases (i.e. enzymes classified under the Enzyme Classification number E.C. 1 (Oxidoreductases) in accordance with the Recommendations (1992) of the International Union of Biochemistry and Molecular Biology (IUBMB)) include oxidoreductases within this group.

Examples include oxidoreductases selected from those classified under the Enzyme Classification (E.C.) numbers:

Glycerol-3-phosphate dehydrogenase (NAD+) (1.1.1.8), Glycerol-3-phosphate dehydrogenase (NAD(P)⁺) (1.1.1.94), Glycerol-3-phosphate 1-dehydrogenase (NADP) (1.1.1.94), Glucose oxidase (1.1.3.4), Hexose oxidase (1.1.3.5), Catechol oxidase (1.1.3.14), Bilirubin oxidase (1.3.3.5), Alanine dehydrogenase (1.4.1.1), Glutamate dehydrogenase (1.4.1.2), Glutamate dehydrogenase (NAD(P)⁺) (1.4.1.3), Glutamate dehydrogenase (NADP⁺) (1.4.1.4), L-Amino acid dehydrogenase (1.4.1.5), Serine dehydrogenase (1.4.1.7), Valine dehydrogenase (NADP⁺) (1.4.1.8), Leucine dehydrogenase (1.4.1.9), Glycine dehydrogenase (1.4.1.10), L-Amino-acid oxidase (1.4.3.2.), D-Amino-acid oxidase(1.4.3.3), L-Glutamate oxidase (1.4.3.11), Protein-lysine 6-oxidase (1.4.3.13), L-lysine oxidase (1.4.3.14), L-Aspartate oxidase (1.4.3.16), D-amino-acid dehydrogenase (1.4.99.1), Protein disulfide reductase (1.6.4.4), Thioredoxin reductase (1.6.4.5), Protein disulfide reductase (glutathione) (1.8.4.2), Laccase (1.10.3.2), Catalase (1.11.1.6), Peroxidase (1.11.1.7), Lipoxygenase (1.13.11.12), Superoxide dismutase (1.15.1.1).

Said glucose oxidases may be derived from Aspergillus niger.

Said laccases may be derived from Polyporus pinsitus, Myceliophtora thermophila, Coprinus cinereus, Rhizoctonia solani, Rhizoctonia praticola, Scytalidium thermophilum and Rhus vernicifera.

Bilirubin oxidases may be derived from Myrothechecium verrucaria.

The peroxidase may be derived from e.g. Soy bean, Horseradish or Coprinus cinereus.

The protein disulfide reductase may be any mentioned in any of the DK patent applications no. 768/93, 265/94 and 264/94 (Novo Nordisk A/S), which are hereby incorporated as reference, including Protein Disulfide reductases of bovine origin, Protein Disulfide reductases derived from Aspergillus oryzae or Aspergillus niger, and DsbA or DsbC derived from Escherichia coli.

Specific examples of readily available commercial oxidoreductases include Gluzyme™ (enzyme available from Novo Nordisk A/S). However, other oxidoreductases are available from others.

The activity of oxidoreductases and variants thereof can be determined as described in “Methods of Enzymatic Analysis”, third edition, 1984, Verlag Chemie, Weinheim, vol. 3.

Carbohydrases

Carbohydrases may be defined as all enzymes capable of hydrolysing carbohydrate chains (e.g. starches) of especially five and six member ring structures (i.e. enzymes classified under the Enzyme Classification number E.C. 3.2 (glycosidases) in accordance with the Recommendations (1992) of the International Union of Biochemistry and Molecular Biology (IUBMB)).

Examples include carbohydrases selected from those classified under the Enzyme Classification (E.C.) numbers:

alpha-amylase (3.2.1.1) beta-amylase (3.2.1.2), glucan alpha-1,4glucosidase (3.2.1.3), cellulase (3.2.1.4), endo-beta-1,3(4)-glucanase (3.2.1.6), endo-beta-1,4-xylanase (3.2.1.8), dextranase (3.2.1.11), chitinase (3.2.1.14), polygalacturonase (3.2.1.15), lysozyme (3.2.1.17), beta-glucosidase (3.2.1.21), alpha-galactosidase (3.2.1.22), beta-galactosidase (3.2.1.23), amylo-1,6-glucosidase (3.2.1.33), xylan beta-1,4-xylosidase (3.2.1.37), glucan endo-beta-1,3-D-glucosidase (3.2.1.39), alpha-dextrin endo-1,6-glucosidase (3.2.1.41), sucrose alpha-glucosidase (3.2.1.48), glucan endo-1,3-alpha-glucosidase (3.2.1.59), glucan beta-1,4-glucosidase (3.2.1.74), glucan endo-beta-1,6-glucosidase (3.2.1.75), arabinan endo-1,5-alpha-arabinosidase (3.2.1.99), lactase (3.2.1.108), chitonanase (3.2.1.132).

Examples of relevant carbohydrases include alpha-1,3-glucanases derived from Trichoderma harzianum; alpha-1,6-glucanases derived from a strain of Paecilomyces; beta-glucanases derived from Bacillus subtilis; beta-glucanases derived from Humicola insolens; beta-glucanases derived from Aspergillus niger; beta-glucanases derived from a strain of Trichoderma; beta-glucanases derived from a strain of Oerskovia xanthineolytica; exo-alpha-1,4-D-glucosidases (glucoamylases) derived from Aspergillus niger; alpha-amylases derived from Bacillus subtilis; alpha-amylases derived from Bacillus amyloliquefaciens; alpha-amylases derived from Bacillus stearothermophilus; alpha-amylases derived from Aspergillus oryzae; alpha-amylases derived from non-pathogenic microorganisms; alpha-galactosidases derived from Aspergillus niger; Pentosanases, xylanases, cellobiases, cellulases, hemi-cellulases deriver from Humicola insolens; cellulases derived from Trichoderma reesei; cellulases derived from non-pathogenic mold; pectinases, cellulases, arabinases, hemi-celluloses derived from Aspergillus niger; dextranases derived from Penicillium lilacinum; endoglucanase derived from non-pathogenic mold; pullulanases derived from Bacillus acidopullyticus; beta-galactosidases derived from Kluyveromyces fragilis; xylanases derived from Trichoderma reesei.

Specific examples of readily available commercial carbohydrases include Alpha-Gal™, Bio-Feed™ Alpha, Bio-Feed™ Beta, Bio-Feed™ Plus, Bio-Feed™ Plus, Novozyme® 188, Carezyme®, Celluclast®, Cellusoft®, Ceremyl®, Citrozym™, Denimax™, Dezyme™, Dextrozyme™, Finizym®, Fungamyl™, Gamanase™, Glucanex®, Lactozym®, Maltogenase™, Pentopan™, Pectinex™, Promozyme®, Pulpzyme™, Novamyl™, Termamyl®, AMG (Amyloglucosidase Novo), Maltogenase®, Sweetzyme®, Aquazym® (all enzymes available from Novo Nordisk A/S). Other carbohydrases are available from other companies.

Lyases

Suitable lyases include Polysaccharide lyases: Pectate lyases (4.2.2.2) and pectin lyases (4.2.2.10), such as those from Bacillus licheniformis disclosed in WO 99/27083.

Isomerases

Examples include isomerases selected from those classified under the Enzyme Classification (E.C.) numbers (5.): e.g. xylose isomerase (5.3.1.5). An example of a relevant isomerase is the Protein Disulfide Isomerase, such as that described in WO 95/01425 (Novo Nordisk A/S). A specific example of a readily available commercial isomerase is Sweetzyme®.

The activity of carbohydrases or variants thereof can be determined as described in “Methods of Enzymatic Analysis”, third edition, 1984, Verlag Chemie, Weinheim, vol. 4.

In another embodiment the test compound is any ingredient, such as stabilizers used for the formulation of enzymes. In particular in relation to the production of enzymes it is of interest to obtain enzyme variants having as little allergenicity as possible in order to reduce the risk of respiratory allergy of the people working in the enzyme industry. Accordingly, when testing for test compounds, such as enzymes and variants thereof, suspected of causing respiratory allergy, it is preferred to use a respiratory epithelial cell.

The test compound may be immunogenic itself or it may be considered as a hapten.

In one embodiment the test compound is any compound of a drug formulation, such as an active drug.

In another embodiment the test compound may be a compound used in cosmetics.

In this case it is particularly contact allergy and a preferred first cell type is a keratinocyte.

Also, the test compound may be an organic solvent, a dye, or a metal.

According to the invention the test compound is added to the cell culture chosen for the purpose in a concentration sufficient to elicit a detectable cytokine response. The concentration of the test compound may vary depending on the test compound in question. In one embodiment of the invention the test compound concentration may be 1, 10 or 100 micrograms/ml. In another aspect the concentration of the test compound added to the cell culture may be dependent on the incubation time of the test compound with the cell culture. In one embodiment of the invention the incubation time of the test compound may be from 0-16 hours, such as 0, 2, 4, 6 or 16 hours.

In another aspect of the present invention the method according to the invention is used for assessing the toxicity of a test compound, comprising the step of:

(a) obtaining a predetermined and precharacterized cell culture comprising at least one cell type of animal, including human, origin capable of substantially non-specific interaction with the test compound, and of responding with a cytokine expression upon interaction,

(b) contacting the cell culture with the test compound,

(c) defining a specific cytokine profiles by determining for at least one predetermined cytokine the cytokine response of the cells exhibiting a substantially non-specific interaction with the test compound, and

(d) correlating the cytokine profile to the toxicity of the test compound.

As specified above with respect to the assessment of the allergenicity of a test compound it is possible to correlate the toxicity of a test compound to the level of cytokines produced, in that the more toxic a compound the lower levels of cytokines are produced. Thus, the invention further relates to a method of simultaneous screening of the toxicity of at least two test compounds, comprising the step of:

(a) arranging a cell culture as defined above in at least two compartments,

(b) contacting the cell cultures individually with a test compound,

(c) defining specific cytokine profiles by determining the cytokine response of the respective cells exhibiting a substantially non-specific interaction with the test compound,

(d) correlating each cytokine profile to the toxicity of the test compounds.

In yet a further aspect the invention relates to an assay kit for assessing the allergenicity or the toxicity for high through-put screening of test compounds, wherein said assay kit comprises as parts of the kit:

(a) a cell culture comprising at least one animal, including human, cell type,

(b) a cytokine determinant selected from at least one pair of monoclonal antibodies with specificity for a specific cytokine, at least one cytokine-specific probe for mRNA detection, at least one set of cytokine-specific primers for mRNA or cDNA detection,

(c) an assay device comprising at least two compartments.

In another embodiment of the invention the assay kit further relates to at least one pair of monoclonal antibodies with specificity for a specific membrane marker, at least one monoclonal antibody with specificity for a specific membrane marker, at least one membrane marker-specific probe for mRNA detection, and at least one set of membrane marker-specific primers for mRNA or cDNA detection.

As described above the assay kit may comprise one cell type or a combination of cell types, the latter either co-cultured or cultured separately. Furthermore, the assay kit may comprise medium for culturing the cells. A person skilled in the art can easily define a medium suitable for culturing the cells specified herein.

Preferably the assay kit comprises an allergenicity standard, which is a standard for assessing the allergenicity of the test compounds in question based on the cytokine profile determined. The allergenicity standard may be in the form of information related to the assay kit, or it may be in the form of co-assessing the test compound with compounds of known allergenicity whereby the cytokine profiles obtained for the test compounds are correlated to the profiles obtained for known compounds.

The assay kit is preferably arranged for testing several test compounds simultaneously, such as testing at least two compounds, more preferably at least 10 test compounds simultaneously, and most preferably capable of assessing the allergenicity of about 100 test compounds simultaneously.

The kit is to be considered as a kit in parts, that is the kit may be combined from several different parts, such as a cell culture from one source, the determinant from another source, and the assay device from a third source.

The assay device is any suitable device, such as a plate with at least one well, wherein the cells may be cultured and contacted with the test compounds.

In a preferred embodiment the assay device comprises at least two compartments in each well, whereby it is possible to co-culture two types of cells in the same well, and assess the cytokine response from the combination as described above. In one embodiment cell cultures may be performed in 24, 48 or 96 well cell culture plates (Nunc). In order to establish co-cultures, where cells are not allowed to have physical contact, specific inserts, figuring well defined permeable membranes, may be used (Nunc TC Inserts).

The assay kit may be used for assessing the allergenicity of any test compound, as well as the toxicity of any test compound.

Experimentals

In the following the methods used in the examples are described.

Methods.

1. Bioassay.

Human lung epithelial cells (BEAS-2B, ATCC # CRL-9609) were inoculated in a culture flask (NUNC), precoated with 2% (vol/vol) Ultroser G in water, at 1500 to 3000 cells per cm², and were grown at 37° C. in LHC-9 medium containing 2% (vol/vol) Ultroser G and 5% CO₂.

The cells were subcultured before reaching confluence. The medium was removed, and fresh 0.5% polyvinylpyrrolidone (PVP) in trypin (0.25% (wt/vol))-EDTA (0.03% (wt/vol)) solution was added until cells detached (usually after 5-10 minutes at room temperature). LHC-9 medium was added, and cells were collected by centrifugation (300× g for 15 minutes), and resuspended in LHC-9 medium containing 2% (vol/vol) Ultroser G. Finally, cells were dispensed into precoated flasks (NUNC) at a density of 1500 to 3000 cells per cm^{2.}

When enough cells were available to perform the planned number of assays, cells were trypinated, and were transferred to a 24-well culture plate (NUNC) (1×10⁵ cells per well). Incubation was for 24 hours at 37° C. in LHC-9 medium containing 2% (vol/vol) Ultroser G and 5% CO₂. Then, the medium was removed and replaced by fresh prewarmed LHC-9 medium containing 2% (vol/vol) Ultroser G and the protein under investigation. Protein-free medium was included as negative control, while cells stimulated with 100 micrograms E. coli LPS 055:B5 were considered as positive controls. Each assay was performed in triplicate.

The extracellular expression of selected cytokines was assessed by ELISA (R&D Systems), as a function of incubation time and protein concentration. ELISA was performed on the cell medium, directly after collection, or on medium that was stored at −20° C. or lower. A calibration curve was included each time, to include quantification of the detected cytokine levels, as well as to assess assay reproducibility.

2. Immunization of Brown Norway rats.

Twenty intratracheal immunizations were performed weekly with 100 microliters 0.9% (wt/vol) NaCl (control group), or 100 microliters of the protein dilution mentioned before. Each group contained 10 rats. Blood samples (2 ml) were collected from the eye one week after every second immunization. Serum was obtained by blood clothing, and centrifugation.

3. IgE ELISA

The IgE ELISA was performed by using the following: Buffers and Solutions:

| | |
|---|---|
| Washing buffer | PBS, 0.05% (v/v) Tween 20 |
| Blocking buffer | PBS, 2% (wt/v) Skim Milk powder |
| Dilution buffer | PBS, 0.05% (v/v) Tween 20, 0.5% (wt/v) |
| | Skim Milk powder |
| Citrate buffer | (0.1 M, pH 5.0-5.2) |

Activation of CovaLink Plates:

Make a fresh stock solution of 10 mg cyanuric chloride per ml acetone.

Just before use, dilute the cyanuric chloride stock solution into PBS, while stirring, to a final concentration of 1 mg/ml. Add 100 microliters of the dilution to each well of the CovaLink NH₂ plates, and incubate for 5 minutes at room temperature. Wash 3 times with PBS.

Dry the freshly prepared activated plates at 50° C. for 30 minutes. Immediately seal each plate with sealing tape. Preactivated plates can be stored at room temperature for 3 weeks when kept in a plastic bag.

ELISA Procedure:

Mouse anti-Rat IgE was diluted 200×in PBS (5 micrograms/ml). 100 microliters were added to each well. The plates were coated overnight at 4° C.

Unspecific adsorption was blocked by incubating each well for 1 hour at room temperature with 200 microliters blocking buffer. The plates were washed 3×with 300 microliters washing buffer.

Unknown rat sera and a known rat IgE solution were diluted in dilution buffer: Typically 10×, 20×and 40×for the unknown sera, and ½ dilutions for the standard IgE starting from 1 microgram/ml. 100 microliters were added to each well. Incubation was for 1 hour at room temperature.

Unbound material was removed by washing 3×with washing buffer. The anti-rat IgE (biotin) was diluted 2000×in dilution buffer. 100 microliters were added to each well. Incubation was for 1 hour at room temperature. Unbound material was removed by washing 3×with washing buffer.

Streptavidin was diluted 1000×in dilution buffer. 100 microliters were added to each well. Incubation was for 1 hour at room temperature. Unbound material was removed by washing 3×with 300 microliters washing buffer.

OPD (0.6 mg/ml) and H₂O₂ (0.4 microliter/ml) was dissolved in citrate buffer. 100 microliters were added to each well. Incubation was for 30 minutes at room temperature.

The reaction was stopped by adding 100 microliters H₂SO₄. The plates were read at 492 nm with 620 nm as reference.

### EXAMPLE 1

Identification of Cytokines that can be Induced in Epithelial Cells

In this set of experiments, 100 micrograms LPS (lipo-polysaccharide) were used to stimulate the cells. ELISA was performed after 2, 4, 6, 8 and 16 hours. Table 2 shows the cytokines induced in the cells (+), and does also indicate the cytokines not detected in the epithelial cell medium when cells are stimulated with LPS (−)

TABLE 2

| Cytokine | Cell response |
|---|---|
| IL-1α | − |
| IL-1β | − |
| IL-2 | − |
| IL-4 | − |
| IL-5 | − |
| IL-6 | + |
| IL-8 | + |
| IFN-γ | − |
| TNF-α | − |
| GM-CSF | + |
| MCP-1 | + |
| MIP-1β | − |
| RANTES | + |

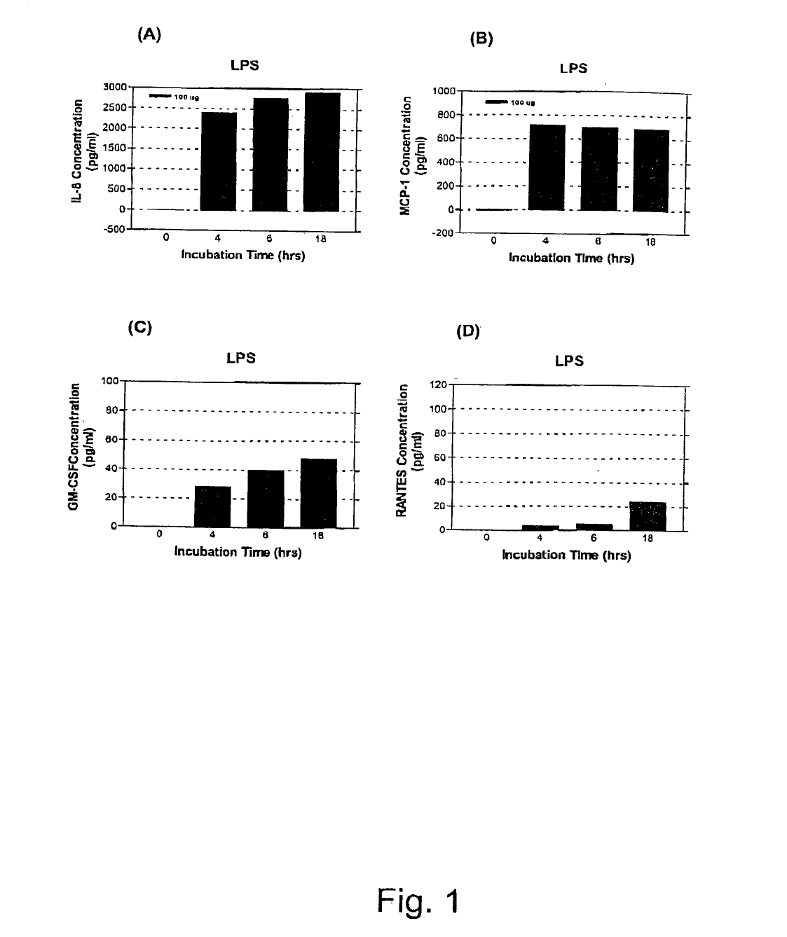

The FIGS. 1A-D show the different kinetics for the cytokines indicated in the table to be positive (+). As IL-8 and IL-6 revealed similar kinetics, only IL-8 is shown (FIG. 1).

### EXAMPLE 2

Assessment of the Cytokine Levels Induced in Epithelial Cells by Various Protease Derivatives

Protease P from the subtilisin family (CDJ31: A Bacillus licheniformis serine protease (E.C. 3.4.21.62)) was selected, and modified with polyethylen-glycol (PEG) molecules of different size, specifically 350, 750, 1000, 2000 and 5000 Da. Both unmodified and modified enzymes were assessed in the epithelial celll assay for there potency to induce extracellular cytokine production.

To prevent proteolysis of the cytokines by the protease, the latter was inhibited by PMSF (10 mM), prior to the dilution in the cell medium. Typically, PMSF was diluted about 1000×before being applied on the cells. At these concentrations, PMSF and its hydrolysis products had no detectable effect on cytokine production.

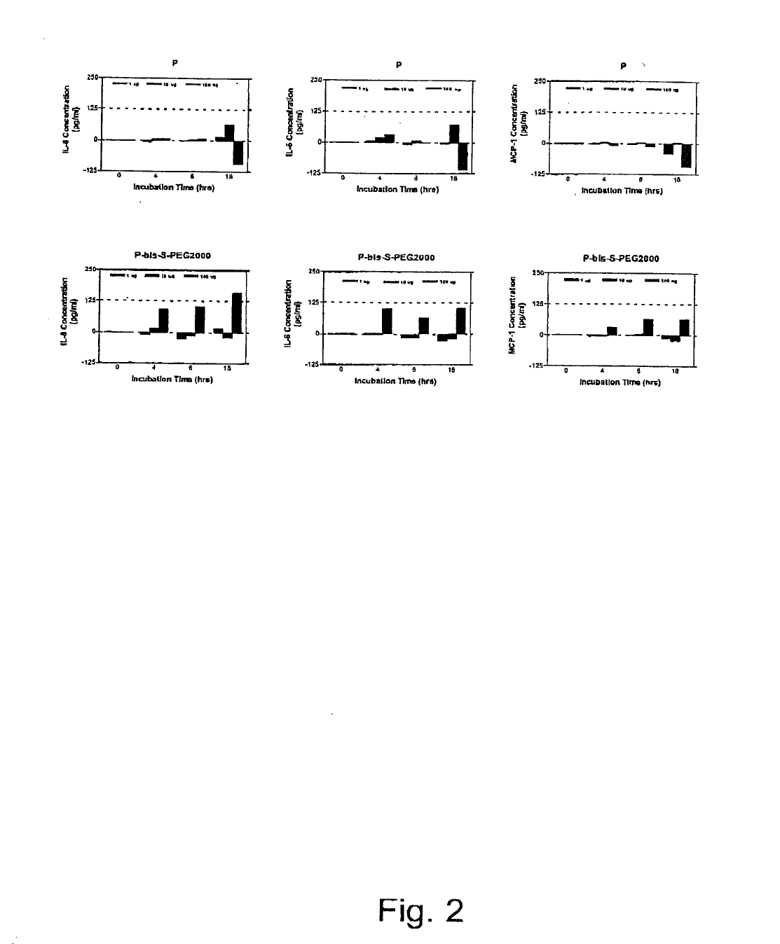

FIG. 2 shows the typically IL-8, IL-6 and MCP-1 kinetics for unmodified and modified protease. The latter is represented by P-bis-S-PEG2000. The baseline is obtained with reference to the control medium. Almost identical results were observed for another subtilisin protease (PD498: A Bacillus sp. Serine protease (E.C. 3.4.21.66)).

For comparison of the respective cytokine levels, the levels resulting after 4 hours of incubation with 100 micrograms of protein were selected.

### EXAMPLE 3

Correlation of the Detected Cytokine Levels and IgE Levels in Rats with the Length of the PEG-Molecules Used for Enzyme Modification

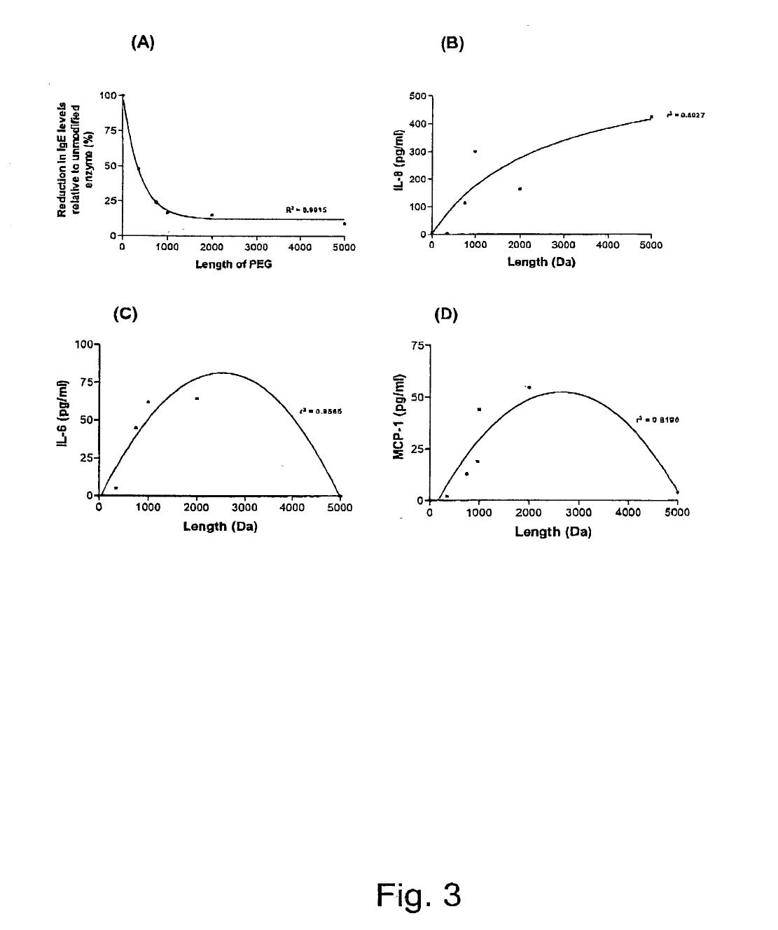

Rats were immunized intratracheally with unmodified and modified protease, and the protease specific IgE levels were detected by ELISA. The IgE levels detected throughout the study were integrated, and compared relative to the levels observed in rats immunized with unmodified enzyme. In FIG. 3, (a) shows a decrease in relative IgE levels with increasing length of the PEG molecules used to modified the enzyme. IL-8 levels (b) were found to increase with increasing PEG size, while both IL-6 (c) and MCP-1 (d) levels revealed bell-shaped kinetics.

### EXAMPLE 4

Correlation of the Detected Cytokine Levels with IgE Levels in Rats as Compared to the Levels Detected for the Unmodified Enzyme

Using the results obtained in Example 3 it is possible directly to correlate the dettected cytokine levels with IgE levels in rats.

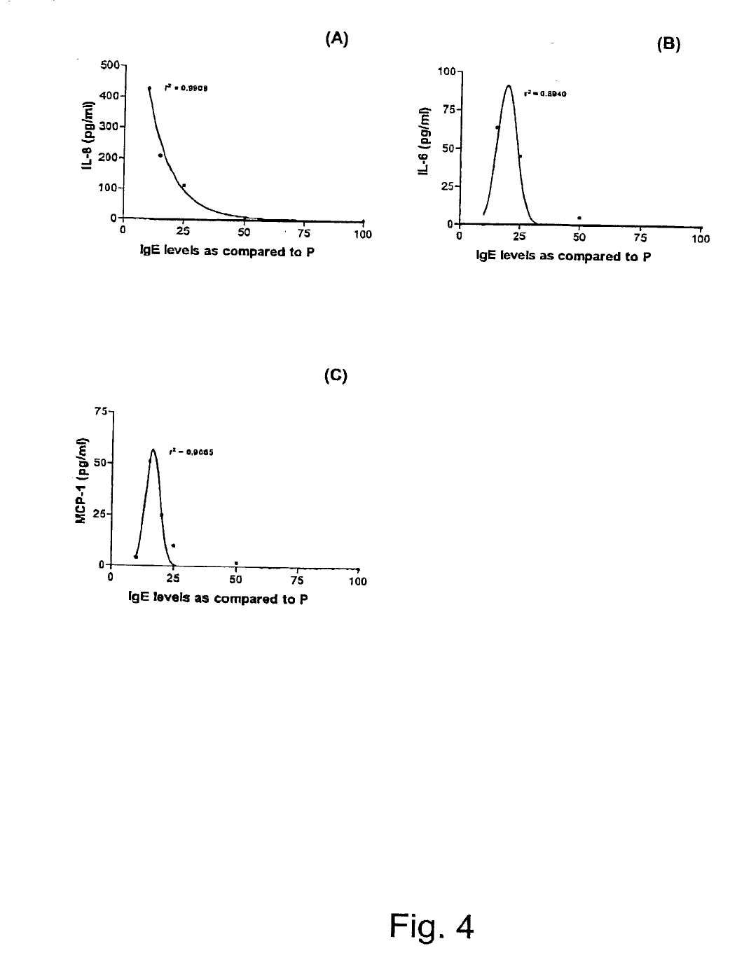

In FIG. 4, (a) shows a decrease in IL-8 levels with increasing IgE levels as compared to the unmodified enzyme. IL-6 (b) and MCP-1 (c) IgE levels steeply increased followed by a steep decline in IgE levels as compared to the unmodified enzyme.

The correlation between IgE and the respective cytokines can be summarized as follows:

TABLE 3

| IgE levels in | | | |
|---|---|---|---|
| rats (%) | IL-8 | IL-6 | MCP-1 |
| >50 | − | − | − |
| 50-25 | + | + | −(p > 0.05) |
| 25-10 | + | + | + |
| <10 | + | − | + |

### EXAMPLE 5

Comparison Between the Detected Cytokine Levels and IgE Levels in Rats of a Protease and Lipolase

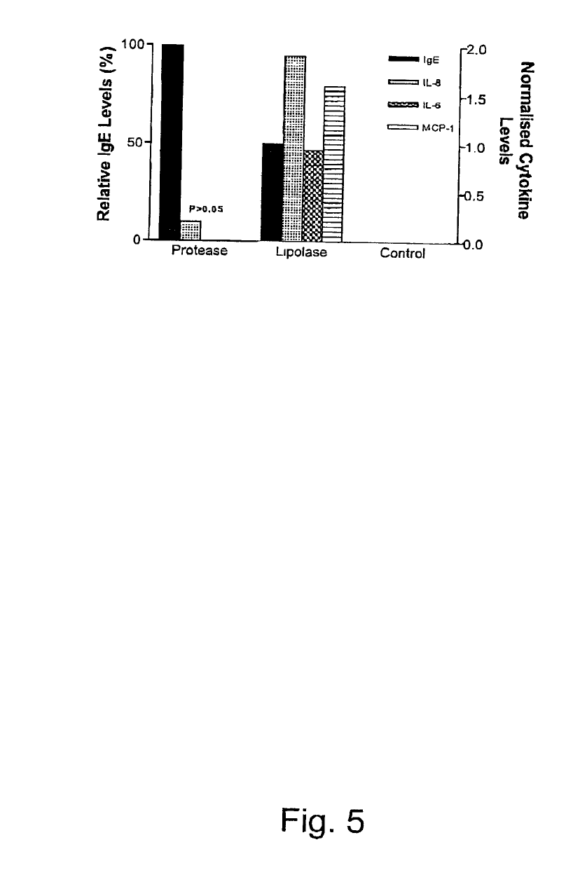

The cytokine levels of serine protease E.C. 3.4.21.66 from Bacillus sp. and triacylglycerol acylhydrolase (lipase) E.C. 3.1.1.3 from Humicola lanuginosa were assessed in the epithelial cell assay. Rats were immunized intratracheally with the same protease and lipase, and the IgE levels were detected by ELISA. FIG. 5 shows the normalized cytokine levels as well as IgE levels of both the protease and the lipase.

Based upon the results shown in table 3 of example 3, the protease would be more allergenic than the lipase. Accordingly, the low level of all cytokines observed for the protease corresponded to a high IgE level, while the high level of all cytokines observed for the lipase corresponded to a significantly lower IgE level.

1. A method for the in vitro assessment of the allergenicity of a test compound, comprising the step of:
(a) obtaining a predetermined and precharacterized cell culture comprising at least one cell type of animal, including human, origin capable of substantially non-specific interaction with the test compound, and of responding with a change in cytokine expression upon interaction,
(b) contacting the cell culture with the test compound,
(c) defining a specific cytokine profile by determining for at least one predetermined cytokine the cytokine response of the cells exhibiting a substantially non-specific interaction with the test compound, and
(d) correlating the cytokine profile to the allergenicity of the test compound.

2. A method for the simultaneous screening of the allergenicity of at least two test compounds, comprising the step of:
(a) arranging a specific cell type in at least two separate compartments of a cell culture of claim 1,
(b) contacting the separated cell culture compartments individually with a test compound,
(c) defining specific cytokine profiles by determining the cytokine responses of the respective cells exhibiting a substantially non-specific interaction with the test compound,
(d) correlating each cytokine profile to the allergenicity of the test compounds.

3. The method of claim 1 or 2, wherein the at least one cell type is epithelial cells.

4. The method of claim 3, wherein the epithelial cells are respiratory tract epithelial cells.

5. The method of claim 3, wherein the epithelial cells are gastro-intestinal tract epithelial cells.

6. The method of claim 1 or 2, wherein the at least one cell type is keratinocytes.

7. The method of claim 1 or 2, wherein the at least one cell type is dendritic cells.

8. The method of claim 1 or 2, wherein the at least one cell type is macrophages.

9. The method of claim 1 or 2, wherein the at least one cell type is mast cells.

10. The method of claim 1 or 2, wherein the at least one cell type is monocytes.

11. The method of claim 1 or 2, wherein the at least one cell type is endothelial cells.

12. The method of any of the preceding claims, comprising the step of:
(a) obtaining at least two cell cultures, each comprising one cell type,
(b) contacting each cell culture with the test compound,
(c) defining specific cytokine profiles by determining the cytokine responses of the respective cell cultures, each exhibiting a substantially non-specific interaction with the test compound,
(d) correlating the cytokine profile to the allergenicity of the test compound.

13. The method of claim 12, wherein the two cell types are co-cultured.

14. The method of claim 12, wherein the cytokine responses of the two cell types are determined for each of the individual cell types.

15. The method of claim 12, wherein the cytokine responses of the two cell types are determined as one response.

16. The method of claim 12, wherein the two cell types of the co-culture are physically separated.

17. The method of any of claims 12-16, wherein one cell type is epithelial cells and the other is dendritic cells.

18. The method of any of claims 12-16, wherein one cell type is epithelial cells and the other is macrophages.

19. The method of any of claims 12-16, wherein one cell type is epithelial cells and the other is mast cells.

20. The method of any of claims 12-16, wherein one cell type is epithelial cells and the other is monocytes.

21. The method of any of claims 12-16, wherein one cell type is epithelial cells and the other is endothelial cells.

22. A method for the in vitro assessment of the toxicity of a test compound, comprising the step of:
(a) obtaining a predetermined and precharacterized cell culture comprising at least one cell type of animal, including human, origin capable of substantially non-specific interaction with the test compound, and of responding with a cytokine expression upon interaction,
(b) contacting the cell culture with the test compound,
(c) defining a specific cytokine profile by determining for at least one predetermined cytokine the cytokine response of the cells exhibiting a substantially non-specific interaction with the test compound, and
(d) correlating the cytokine profile to the toxicity of the test compound.

23. A method for the simultaneous screening of the toxicity of at least two test compounds, comprising the step of:
(a) arranging a cell culture of claim 22 in at least two compartments,
(b) contacting the cell cultures individually with a test compound,
(c) defining specific cytokine profiles by determining the cytokine responses of the respective cells exhibiting a substantially non-specific interaction with the test compound,
(d) correlating each cytokine profile to the toxicity of the test compounds.

24. The method of any of claims 1-23, wherein the test compound is a protein.

25. The method of any of claims 1-23, wherein the test compound is a glycoprotein.

26. The method of any of claims 1-23, wherein the test compound is a lipoprotein.

27. The method of any of claims 1-23, wherein the test compound is a proteolipid.

28. The method of any of claims 1-23, wherein the test compound is a phospholipid.

29. The method of any of claims 24-28, wherein the test compound is an enzyme or an enzyme variant.

30. The method of any of claims 1-29, wherein the test compound is for the formulation of enzymes.

31. The method of any of claims 1-21, wherein the test compound is a hapten.

32. The method of any of claims 1-31, wherein the test compound is a drug.

33. The method of claim 31, wherein the test compound is a cosmetic compound.

34. The method of claim 22 or 23, wherein the test compound is an organic solvent.

35. The method of claim 31, wherein the test compound is a dye.

36. The method of claim 31, wherein the test compound is a metal.

37. The method of any of the preceding claims, wherein the at least one cell type is derived from human tissue.

38. The method of any of claims 1-37, wherein the at least one cell type is derived from human blood.

39. The method of any of the preceding claims, wherein the extracellular cytokine response is determined by analysis with enzyme-linked immunosorbent assay.

40. The method of any of claims 1-38, wherein the intracellular cytokine response is determined by analysis with enzyme-linked immunosorbent assay.

41. The method of any of claims 1-38, wherein the cytokine response is determined by in-situ-hybridization technique.

42. The method of any of claims 1-38, wherein the cytokine response is determined by analysis with polymerase-chain-reaction (PCR) technique.

43. The method of claim 42, wherein the cytokine response is determined by analysis with quantitative-PCR technique.

44. The method of claim 42, wherein the cytokine response is determined by analysis with in-situ-PCR technique.

45. The method of any of claims 41-44, wherein intracellular mRNA is determined.

46. The method of claim 1 or 22, wherein membrane markers induced by the test compound in cells exhibiting a substantially non-specific interaction with the test compound, are determined.

47. The method of claim 46, wherein the membrane markers assayed for are VCAM-1 or ICAM-1.

48. The method of any of the preceding claims, wherein more than one cytokine is being assayed for.

49. The method of claim 48, wherein at least two cytokines are being assayed for.

50. The method of claim 48, wherein at least four cytokines are being assayed for.

51. The method of claim 48, wherein the cytokines assayed for are interleukin-8, interleukin-6, MCP-1, and GM-CSF.

52. The method of claim 1, wherein the level and number of cytokines assessed are factors correlating to the allergenic potency of the test compound.

53. The method of claim 52, wherein the level and number of cytokines assessed are factors correlating to low allergenic test compounds.

54. The method of claim 52, wherein the level and number of cytokines assessed are factors correlating to high allergenic test compounds.

55. The method of claim 23, wherein the level and number of cytokines assessed are factors correlating to the toxic potency of the test compound.

56. The method of claim 55, wherein the level and number of cytokines assessed are factors correlating to low toxic test compounds.

57. The method of claim 55, wherein the level and number of cytokines assessed are factors correlating to high toxic test compounds.

58. A method of characterizing a cytokine response of a cell type as described in any of the preceding claims, comprising the step of:
(a) obtaining a predetermined cell culture comprising one cell type of animal, including human, origin capable of substantially non-specific interaction with the test compound, and of responding with a cytokine expression upon interaction,
(b) identifying the cytokines that can be expressed by the cell culture upon non-specific interaction with the test compound by using a non-specific multivalent inducer,
(c) contacting a part of the cell culture with a test compound, determining the level of the identified cytokines obtaining a cytokine profile,
(d) immunizing an animal with the test compound and determining the resulting IgE level of the animal,
(e) correlating the cytokine profile to the IgE level determined, and
(f) repeating step c)-e) until at least a test compound of a high IgE level, a test compound of a low IgE level and a test compound of a medium IgE level have been tested.

59. An assay kit for high through-put screening of the allergenicity or toxicity of a test compound, comprising:
(a) a cell culture comprising at least one animal, including human, cell type,
(b) a cytokine determinant selected from at least one monoclonal antibody with specificity for a specific cytokine, at least one cytokine-specific probe for mRNA detection, at least one set of cytokine-specific primers for mRNA or cDNA detection,
(c) an assay device comprising at least two compartments.

60. The assay kit as described in claim 59, comprising at least one monoclonal antibody with specificity for a specific membrane marker, at least one monoclonal antibody with specificity for a specific membrane marker, at least one membrane marker-specific probe for mRNA detection, and at least one set of membrane marker-specific primers for mRNA or cDNA detection.

61. The assay kit of claim 59 or 60, further comprising an allergenicity standard.

62. The use of an assay kit of any of claims 59-61 for the high through-put screening of the allergenicity of at least two test compounds.

63. The use of an assay kit of claim 62 for assessing the allergenicity of at least 10 test compounds simultaneously.

64. The use of claim 62 for assessing the allergenicity of about 100 test compounds simultaneously.

65. The use of an assay kit of claim 60 for the high through-put screening of the toxicity of at least two test compounds.

66. The use of an assay kit of claim 65 for assessing the toxicicity of at least 10 test compounds simultaneously.

67. The use of claim 65 for assessing the toxicity of about 100 test compounds simultaneously.
